(19) United States
(12) Patent Application Publication
(10) Pub. No.: US 2023/0272152 A1
(43) Pub. Date: Aug. 31, 2023

(54) NOVEL POLYURETHANES AND THEIR USE IN PHARMACEUTICAL DOSAGE FORMS

(71) Applicant: BASF SE, Ludwigshafen (DE)

(72) Inventors: Ferdinand Paul Brandl, Ludwigshafen (DE); Theo Smit, Ludwigshafen (DE); Karl Kolter, Limburgerhof (DE); Felicitas Guth, Ludwigshafen (DE); Karl Haeberle, Speyer (DE); Frederic Lucas, Ludwigshafen (DE)

(21) Appl. No.: 18/029,972

(22) PCT Filed: Oct. 5, 2021
(86) PCT No.: PCT/EP2021/077330

§ 371 Date: Apr. 3, 2023

(30) Foreign Application Priority Data
Oct. 7, 2020 (EP) 20200535.1

(51) Int. Cl. C08G 18/34; C08G 18/12; A61K 9/16; C08G 18/32

(52) U.S. Cl. CPC C08G 18/348; A61K 9/1641; C08G 18/12; C08G 18/3206; C08G 18/3212; C08G 18/3218

(57) ABSTRACT

The present invention relates to novel polyurethanes based on a diisocyanate, a carboxylic acid functionalized diol and an acid-free diol, their use as pharmaceutical excipients for improving gastrointestinal absorption, the respective pharmaceutical dosage forms and methods for making the polyurethanes.

The present invention relates to novel polyurethanes based on a diisocyanate, a carboxylic acid functionalized diol and an acid-free component containing at least two hydroxy groups, their use as pharmaceutical excipients for improving gastrointestinal absorption, the respective pharmaceutical dosage forms and methods for making the polyurethanes.

The intestinal absorption of poorly water-soluble drugs (BCS class II and IV) is limited by the maximum achievable concentration in the gastrointestinal lumen. Therefore, various approaches in formulation development aim at increasing the dissolution rate and improving drug solubility in the gastrointestinal tract. Administering the drug as a solution is a common approach to enhance the intestinal absorption of poorly water-soluble drugs. To this end, hydrophobic drugs are formulated using a mixture of co-solvents, surfactants, complexing agents (e.g., cyclodextrins) and/or oils. After oral administration, these formulations increase the total concentration of the drug that is present in solution; however, this approach does not necessarily result in an improved bioavailability. Depending on the lipophilicity of the drug, a large fraction of drug molecules is solubilized in a mixture of colloidal species (e.g., emulsified oil, micelles etc.). This fraction is unavailable for absorption, since only the free molecular species of the drug can permeate across the intestinal barrier. Furthermore, dilution and dispersion of the formulation in the gastrointestinal tract decreases the solubilization capacity. As a result, a metastable supersaturated state is generated that eventually leads to drug precipitation.

Besides the administration in solution, a number of formulation strategies exist that enable the delivery of poorly water-soluble drugs in a solid form. These approaches aim at generating high-energy or rapidly dissolving forms of the drugs (e.g., by milling, co-grinding, solvent evaporation, melting or crystal engineering) that induce supersaturation in the gastrointestinal tract. For example, in combination with suitable polymers (e.g., polyvinylpyrrolidone, vinylpyrrolidone-vinyl acetate copolymer, polyethylene glycol, polymethacrylates, cellulose derivatives etc.) and/or surfactants, poorly water-soluble drugs can be manufactured into solid dispersions (e.g., by spray drying or hot melt extrusion). These contain amorphous drug particles embedded in a polymer matrix that stabilizes the amorphous state by vitrification, specific drug-polymer interactions and/or reduced mobility. The release of the embedded drug molecules often depends on the dissolution rate of the polymer matrix. After dissolution of the dosage form in the gastrointestinal tract, the concentration of the drug in solution will be above the saturation solubility. This supersaturated state is thermodynamically unstable, and the system tends to return to the equilibrium state by drug precipitation. To benefit from the increased concentration, it is necessary to stabilize the supersaturated state in the gastrointestinal lumen for a time period sufficient for absorption to take place. Polymers can inhibit drug precipitation by interfering with nucleation and/or crystal growth. It is important to note that this type of stabilization in solution is different from the stabilization of the amorphous state in the dosage form prior to application.

WO2014/159748 mentions the use of polyacrylate based crystallization-inhibiting agents, preferably a copolymer of butyl methacrylate, 2-dimethylaminethyl methacrylate and methyl methacrylate in a weight ratio 1:2:1.

WO 2005/058383 describes adhesive implants for parietal repair comprising water-soluble biocompatible polymers having adhesive properties which are copolymers based on alkyl acrylates such as octyl acrylates as well as acrylic acid and hydroxyalkyl (meth)acrylates.

WO 2014/182713 relates to statistical copolymers made from at least three different acrylate monomers such as alkyl(meth)acrylate, carbalkoxyalkyl (meth)acrylates, hydroxyalkyl (meth)acrylates and alkyl acetyl acrylates and their use for inhibiting drug crystallization and supersaturation maintenance. WO 2014/182710 refers to similar copolymers further substituted with sugar moieties.

The acrylic terpolymers described in WO 2019/121051 are based on acrylic acid, a hydrophobic methacrylate and a third olefinic monomer selected from the group consisting of N-vinyl lactams, 2-hydroxyethyl methacrylate and phenoxyethyl acrylate, and inhibit drug crystallization in aqueous solutions.

Of the cellulose derivatives, hydroxypropyl methyl cellulose acetate succinate (HPMCAS) is considered to be the most effective polymer to inhibit drug precipitation [J. Brouwers, M.E. Brewster, P. Augustijns, Supersaturating drug delivery systems: The answer to solubility-limited oral bioavailability? Journal of Pharmaceutical Sciences, 98 (2008) 2549-2572; D.B. Warren, H. Benameur, C.J.H. Porter, C.W. Pouton, Using polymeric precipitation inhibitors to improve the absorption of poorly water-soluble drugs: A mechanistic basis for utility, Journal of Drug Targeting, 18 (2010) 704-731; S. Baghel, H. Cathcart, N.J. O′Reilly, Polymeric amorphous solid dispersions: A review of amorphization, crystallization, stabilization, solid-state characterization, and aqueous solubilization of biopharmaceutical classification system class II drugs, Journal of Pharmaceutical Sciences, 105 (2016) 2527-2544].

The number of drugs suffering from poor solubility is increasing [A.M. Thayer, Finding Solutions, Chem. Eng. News 88 (2010) 13-18; P.D. Leeson, Molecular inflation, attrition and the rule of five, Advanced Drug Delivery Reviews 101 (2016) 22-33]. Considering that one polymer product will not be an effective inhibitor for all drug molecules [G.A. Ilevbare, H. Liu, K. J. Edgar, L.S. Taylor, Maintaining Supersaturation in Aqueous Drug Solutions: Impact of Different Polymers on Induction Times, Crystal Growth & Design, 13 (2013) 740-751], the large variety of drug structures also requires a variety of different polymers that can be used to stabilize supersaturated solutions. The problem to be solved by the present invention was to identify a crystallization inhibiting polymer that is structurally different from the known vinyl, acrylic and cellulose based inhibitors and that his highly effective in stabilizing supersaturated solutions of range of different drugs. The problem was solved by finding polyurethanes based on a diisocyanate, a carboxylic acid functionalized diol and a carboxylic acid-free diol.

The use of polyurethane drug delivery systems has been reported. In these systems, the polyurethane functions as an insoluble matrix from which the drug is released over time by degradation or swelling of the polyurethane matrix. Examples of such systems are implants, inserts and drug carrier particles [C. Englert, J.C. Brendel, T.C. Majdanski, T. Yildirim, S. Schubert, M. Gottschaldt, N. Windhab, U.S. Schubert, Pharmapolymers in the 21st century: Synthetic polymers in drug delivery applications, Progress in Polymer Science 87 (2018) 107-164; J.Y. Cherng, T.Y. Hou, M.F. Shih, H. Talsma, W. E. Hennink, Polyurethane-based drug delivery systems, International Journal of Pharmaceutics 450 (2013) 145- 162; B. Claeys, A. Vervaeck, X.K.D. Hillewaere, S. Possemiers, L. Hansen, T. De Beer, J.P. Remon, C. Vervaet, Thermoplastic polyurethanes for the manufacturing of highly dosed oral sustained release matrices via hot melt extrusion and injection molding, European Journal of Pharmaceutics and Biopharmaceutics 90 (2015) 44-52; G. Verreck, I. Chun, J. Rosenblatt, J. Peeters, A. Van Dijck, J. Mensch, M. Noppe, M.E. Brewster, Incorporation of drugs in an amorphous state into electrospun nanofibers composed of a water-insoluble, nonbiodegradable polymer, Journal of Controlled Release 92 (2003) 349-360; M.R. Nabid, I. Omrani, Facile preparation of pH-responsive polyurethane nanocarrier for oral delivery, Materials Science and Engineering C 69 (2016) 532-537; B.S. Eftekhari, A. Karkhaneh, A. Alizadeh, Physically Targeted Intravenous Polyurethane Nanoparticles for Controlled Release of Atorvastatin Calcium, Iranian Biomedical Journal 21 (2017) 369-379; A. Y. Khosroushahi, H. Naderi-Manesh, H. Yeganeh, J. Barar, Y. Omidi, Novel water-soluble polyurethane nanomicelles for cancer chemotherapy, Journal of Nanobiotechnology 10 (2012)]. In these listed reports, after drug release, the dissolved drug and the insoluble polyurethane no longer interact. The polymer in these cases has therefore no influence on the solution behavior of the dissolved drug. The use of a soluble polyurethane to stabilize a solution of a poorly soluble drug against crystallization is new.

The inventive polyurethanes comprise components A to D with 45-70 wt% of at least one diisocyanate A with at least one ring in the molecular structure between the two isocyanate groups; with 15-40 wt% of at least one component B with i) two primary hydroxy groups, ii) one secondary or tertiary carboxylic acid group iii) a molecular weight between 100 and 250 g/mol and iv) no additional groups that are reactive towards isocyanates; with 5-30 wt% of at least one component C with i) at least two hydroxy groups, ii) a molecular weight between 60 and 250 g/mol, iii) no acid groups and iv) no primary or secondary amine or thiol groups and optionally with 0-5 wt% of one or more components D which i) contain one or two groups reactive toward isocyanate groups and ii) maximum one of these reactive groups is a hydroxy group.

In another embodiment the inventive polyurethanes are essentially consisting of components A to D with 45-70 wt% of at least one diisocyanate A with at least one ring in the molecular structure between the two isocyanate groups; with 15-40 wt% of at least one component B with i) two primary hydroxy groups, ii) one secondary or tertiary carboxylic acid group iii) a molecular weight between 100 and 250 g/mol and iv) no additional groups that are reactive towards isocyanates; with 5-30 wt% of at least one component C with i) at least two hydroxy groups, ii) a molecular weight between 60 and 250 g/mol, iii) no acid groups and iv) no primary or secondary amine or thiol groups and optionally with 0-5 wt% of one or more components D which i) contain one or two groups reactive toward isocyanate groups and ii) maximum one of these reactive groups is a hydroxy group.

In a further embodiment the inventive polyurethanes are consisting of components A to D with 45-70 wt% of at least one diisocyanate A with at least one ring in the molecular structure between the two isocyanate groups; with 15-40 wt% of at least one component B with i) two primary hydroxy groups, ii) one secondary or tertiary carboxylic acid group iii) a molecular weight between 100 and 250 g/mol and iv) no additional groups that are reactive towards isocyanates; with 5-30 wt% of at least one component C with i) at least two hydroxy groups, ii) a molecular weight between 60 and 250 g/mol, iii) no acid groups and iv) no primary or secondary amine or thiol groups and optionally with 0-5 wt% of one or more components D which i) contain one or two groups reactive toward isocyanate groups and ii) maximum one of these reactive groups is a hydroxy group. The total amount of incorporated components A to D adds up to 100% by weight. The inventive polymers are synthesized from A to D in a polyaddition process. Residual isocyanate groups are hydrolyzed after the polyaddition process. The polymer product is therefore free of isocyanate groups. At least 25% of the carboxylic acid groups of B are neutralized with a base before, during or after the polymerization.

Another aspect of the invention is the use of the polyurethanes for inhibiting in vivo recrystallization of an active ingredient after release from a dosage form into the aqueous environment of the human or animal body and the respective dosage forms comprising the copolymer and an active ingredient, wherein the active ingredient has a solubility in water under standard conditions (temperature of 23° C. and a pressure of 0.101325 MPa) of less than 0.1 % by weight. Preferably, the solubility of the active ingredient in water under standard conditions is less than 0.05 % by weight, the active ingredient being present in such dosage form in an amorphous state or molecularly dispersed. Amorphous means that less than 5 % by weight are crystalline. The crystalline proportion can be measured by X-Ray diffraction methods.

In accordance with the present invention solubility whether in water, phosphate buffer or other suitable biologically relevant systems is always the solubility at standard conditions, i.e., a temperature of 23° C. and a pressure of 0.101325 MPa.

According to the invention, active ingredients sparingly soluble in water are those having a solubility of less than 0.1 % by weight in water at standard conditions.

In all embodiments of the invention the amounts for the monomer derived moieties given in percent by weight are meant to include a deviation of ± 1 % by weight.

The polymers can be prepared in a conventional manner in a polyaddition process. The polymerization can be carried out in bulk or in solution in aprotic organic solvents. Examples of organic solvents include ketones such as acetone, methyl ethyl ketone, methyl isobutyl ketone, and cyclohexanone; nitriles such as acetonitrile; alkyl esters such as ethyl acetate and butyl acetate, aromatic hydrocarbons such as toluene and xylene, ethers such as diethyl ether, tetrahydrofuran, and dioxane; aprotic polar solvents such as N-methylpyrrolidone, dimethylformamide, N,N′-dimethyl acetamide and dimethyl sulfoxide.

All the components can be included in the initial reactor charge. It is also possible, to first allow a part of the components to react and to add the remaining monomers at a later point in time. The components can be added at once to the reactor or can be fed into the reactor over a longer period of time. The reaction time may be in the range from a few hours to several days.

The polyaddition reaction can be accelerated by the use of suitable catalysts such as tertiary amines and organometallic compounds. Examples of tertiary amines catalysts are diazabicyclo[2.2.2]octane, 2-ethyl-4-methylimidazol, 1,8-diazabicyclo[5.4.0]undec-7-ene and Nmethylmorpholine. Examples of organometallic catalysts are dibutyltin dilaurate and tin(II) octoate.

The polymerization may be conducted at temperatures from 20 to 180° C., preferably from 50 to 130° C. The polymerization can be carried out both under atmospheric pressure or in a closed reactor under elevated pressure. In this case, it is possible to polymerize either under the pressure set up during the reaction, or the pressure can be adjusted by injecting a gas or evacuating.

Neutralization of carboxylic acid groups ensures that the polymer is able to dissolve in an aqueous environment. Once dissolution has occurred, the degree of neutralization of the polymer carboxylic acid groups will be determined by the pH of the medium. The inventive polyurethanes were found to be only moderately hygroscopic. A polyurethane synthesized from 60 wt% isophorone diisocyanate (Component A), 27 wt% 2,2-bis(hydroxymethyl)butyric acid (Component B) and 13 wt% 1,4-cyclohexane dimethanol (Component C), with 90% of the carboxylic acid groups neutralized with sodium hydroxide, adsorbs 12% moisture upon storage at 70% relative humidity at 25° C. In contrast, a polyacrylate synthesized from 20 wt% N-vinylpyrrolidon, 55 wt% t-butyl methacrylate and 25 wt% acrylic acid, also with 90% of the carboxylic acid groups neutralized with sodium hydroxide, was found to adsorb 26% moisture under identical conditions.

According to the invention suitable examples of diisocyanates A are toluene diisocyanate (2,4-or 2,6-toluene diisocyanate or a mixture thereof) (TDI), hydrogenated TDI (H6TDI), 1,5-naphthalene diisocyanate (NDI), 3,3′-Dimethylbiphenyl-4,4′-diisocyanatemethylene (TODI), diphenyl diisocyanate (4,4′-, 2,4′- or 2,2′- methylene diphenyl diisocyanate or a mixture thereof) (MDI), xylylene diisocyanate (1,3- or 1,4-xylylene diisocyanate or a mixture thereof) (XDI), tetramethylxylylene diisocyanate (1,3- or 1,4-tetramethylxylylene diisocyanate or a mixture thereof) (TMXDI), 1,3-cyclopentane diisocyanate, 1,3-cyclopentene diisocyanate, cyclohexane diisocyanate (1,4-cyclohexane diisocyanate (CHDI), 1,3-cyclohexane diisocyanate), 3-isocyanatomethyl-3,5,5-trimethylcyclohexyl isocyanate (isophorone diisocyanate) (IPDI), methylene bis(cyclohexylisocyanate)(4,4′-, 2,4′- or 2,2′-methylene bis(cyclohexyl isocyanate, transtrans isomer, trans-cis isomer, cis-cis isomer or a mixture thereof) (H₁₂MDI), methylcyclohexane diisocyanate (methyl-2,4-cyclohexane diisocyanate, methyl-2,6-cyclohexane diisocyanate) or bis(isocyanatomethyl) cyclohexane (1,2-, 1,3- or 1,4-bis(isocyanatomethyl) cyclohexane) (H₆XDI). Other suitable compounds are variations of the listed diisocyanates which in addition or instead of free isocyanate groups have functional groups which liberate isocyanate groups or react like isocyanate groups. Examples of these are compounds having capped isocyanate groups or uretdione groups. Capped isocyanate groups are produced during reaction with a blocking agent, which liberates the isocyanate groups when the blocked isocyanate groups are heated to a temperature at least equal to what is known as the deblocking temperature. Examples of compounds which block (cap or protect) isocyanate groups are caprolactam, imidazoles, malonic esters, dialkylamines or oximes. In the case of capped isocyanates, the 45-70 wt% of diisocyanate for the synthesis of the inventive polymers refers to the amount of free diisocyanate that can be obtained from the capped diisocyanate. Preferred diisocyanates are TMXDI and IPDI, whereby IPDI is most preferred. All of the listed diisocyanates may be used singly or in combination of two or more.

Examples of component B include N,N-bishydroxyethyl alanine, 2,2-bis(hydroxymethyl)acetic acid, 2,2-bis(hydroxymethyl)propionic acid (DMPA), 2,2-bis(hydroxyethyl)propionic acid, 2,2-bis(hydroxymethyl)butyric acid (DMBA) or 2,2-di(hydroxymethyl)pentanoic acid. Of these compounds, 2,2-bis(hydroxymethyl)propionic acid (DMPA) and 2,2-bis(hydroxymethyl)butyric acid (DMBA) are preferred, whereby DMBA is the most preferred. The listed diols B may be used singly or in combination of two or more. The carboxylic acid can be protected by a group that can be removed in a post-polymerization step to regain the carboxylic acid or the neutralized carboxylic acid. The carboxylic acid can for example be protected by esterification.

At least 25% of the carboxylic acid groups of the inventive polyurethanes are neutralized (deprotonated) by a base. The carboxylic acid can be neutralized before, during or after the polymerization reaction. Suitable neutralizing agents are alkali metal bases, such as sodium hydroxide, potassium hydroxide, sodium carbonate, sodium hydrogen carbonate, potassium carbonate, or potassium hydrogen carbonate, amines, such as triethylamine, triethanolamine or meglumine, basic amino acids, such as lysine and arginine, and ammonia. Sodium hydroxide is preferred. In the case of amine bases and ammonia, the carboxylic acid groups are neutralized after the isocyanate groups have been converted.

Component C does not contain acid groups. Acid refers to the Brønsted-Lowry definition of an acid, namely compounds that can donate a proton. Examples are carboxylic acids, sulfonic acids and phosphonic acids.

Examples of component C include 1,4-butanediol, 1,6-hexanediol, 1,3-cyclopentanediol, 2,5-bis(hydroxymethyl)furan, 5-norbornene-2,2-dimethanol, 5-norbornene-2-exo,3-exo-dimethanol, 5-norbornene-2-endo,3-endo-dimethanol, 3,4-bis(hydroxymethyl)furan, 1,3-cyclopentane dimethanol, 1,3- and 1,4-cyclohexanediol, 1,3- and 1,4-cyclohexane dimethanol, 2,2-diethyl-1,3-propanediol, 2,5-dimethyl-2,5-hexanediol, isosorbide, fructose, xylose, sorbitol, mannitol, neopentyl glycol and hydrogenated bisphenol A. Preferred are 2,2-diethyl-1,3-propanediol, 2,5-bis(hydroxymethyl)furan, 2,5-dimethyl-2,5-hexanediol, neopentyl glycol, isosorbide and 1,3- and 1,4-cyclohexane dimethanol. Most preferred are 2,2-diethyl-1,3-propanediol, 2,5-dimethyl-2,5-hexanediol, neopentyl glycol and 1,4-cyclohexane dimethanol.

In addition to components A-C, the polymers used according to the invention may incorporate maximum 0-5 wt% of one or more of components D. Component D contains one or two groups that are reactive towards isocyanate groups, selected from hydroxy groups, primary and secondary amino groups, and thiol groups. Depending on these groups, the resultant polymers have urethane groups, urea groups, and/or thiocarbamate groups. Component D differs from components B and C in that maximum one of these groups is a hydroxy group. Component D can for example be used as a chain terminating agent, a chain extender, or to introduce urea groups into the polyurethane. Examples of component D are diamines such as 1,2-, 1,3- and 1,4-diaminocyclohexane, isophorone diamine, 4,4′-diaminodicyclohexylmethane, neopentanediamine, 1,3- and 1,4-bis(aminomethyl)-cyclohexane, 2,5-bis(aminomethyl)furan, amino alcohols such as 2-aminoethanol, 2-(N-methylamino)ethanol, 3-aminopropanol, 2-amino-2-methyl-1-propanol, monofunctional alcohols, such as methanol, ethanol, n-propanol, isopropanol, and amines having one primary or secondary amino group such as isopropylamine and diisopropylamine, and amino acids such as glycine and alanine. The molecular weight of these compounds is preferably between 60 and 300 g/mol.

The weight average molecular weight (M_{w}) of the inventive polymers, measured by gel permeation chromatography, using poly(methyl methacrylate) standards, lies in the range of 2,000 to 100,000 g/mol, preferably 3,000 to 70,000 g/mol and most preferably 4,000 to 60,000 g/mol.

In an embodiment of the invention the polyurethane comprises only components A to C with 50-65 wt% of isophorone diisocyanate as A; with 20-35 wt% of at least one component B, selected from the group consisting of 2,2-bis(hydroxymethyl)butyric acid and 2,2-bis(hydroxymethyl)propionic acid, and with 5-30 wt% of at least one component C selected from the group consisting of 1,4-cyclohexane dimethanol, 1,3-cyclohexane dimethanol, 2,2-diethyl-1,3-propanediol, 2,5-dimethyl-2,5-hexanediol, isosorbide and neopentyl glycol, whereby the total amount of incorporated components A to C adds up to 100% by weight.

Groups reactive toward isocyanate groups are preferably those selected among hydroxy groups, primary and secondary amino groups, and thiol groups. Depending on these groups, the resultant polymers have urethane groups, urea groups, and/or thiocarbamate groups.

After the polyaddition reaction, a large excess of water with respect to the amount of residual isocyanate groups is added to the reaction mixture to hydrolyze residual isocyanates groups. The inventive polymers are free of isocyanates. In this they differ from so called polyurethane prepolymers with isocyanate end groups. These polymers are prepared using a significant excess of isocyanate over groups that are reactive towards isocyanates. Alternatively, it is also possible to use an excess of alcohol groups to obtain a prepolymer with hydroxy end groups. Prepolymers are made for the purpose of being converted into the final product in a subsequent reaction. (H-W. Engels, H-G. Pirkl, R. Albers, R. W. Albach, J. Krause, A. Hoffmann, H. Casselmann, J. Dormish, Polyurethanes: Versatile Materials and Sustainable Problem Solvers for Today’s Challenges, Angewandte Chemie International Edition, 52 (2013) 9422 - 9441). WO2019035382 describes hollow resin particles to be used as heat-sensitive recording material. Polymers are described that are prepared from a carboxyl group-containing active hydrogen group-containing component, a polyisocyanate component and a second active hydrogen group-containing component. Examples describe polymers prepared from isophorone diisocyanate, 1,4-cyclohexane dimethanol and 2,2-bis(hydroxymethyl)propionic acid. The highest amount of 2,2-bis(hydroxymethyl)propionic acid on the total amount of monomer in these examples is 6.0 wt%. These polymers differ from the polymers of the invention described herein, firstly in the lower amount of carboxylic acid diol (maximum 6.0 wt%) and secondly in the fact that the polymers in WO2019035382 are prepolymers that are prepared using an excess of isocyanate (isocyanate/ hydroxy ratio is 2:1) which results in the formation of a polymer with isocyanate end groups. CN103539914 describes heat-resistant polyurethane resin for coating applications. Polymer solutions are prepared from 5-20 wt% 2,2-bis(hydroxymethyl)propionic acid and 2,2 bis(hydroxymethyl)butyric acid, 20-40 wt% polyisocyanate, 5-20 wt% heat-resistant diol and 30-60 wt% solvent). Examples of CN103539914 do not describe how much of the different monomers were used (ranges are given) but it is stated that the synthesized product is a linear prepolymer with a hydroxy group at both chain ends. The prepolymer is converted in a subsequent reaction. Prepolymers with hydroxy end groups are obtained by using an excess of hydroxy groups over isocyanate groups. In this the prepolymers of CN103539914 differ from the polymers of the invention described herein.

According to the invention the active ingredients can be selected from the group of pharmaceutical, nutritional or agrochemical actives.

Examples which may be mentioned here include antihypertensives, vitamins, cytostatics, especially taxol, anesthetics, neuroleptics, antidepressants, antibiotics, antimycotics, fungicides, chemotherapeutics, urologics, platelet aggregation inhibitors, sulfonamides, spasmolytics, hormones, immunoglobulins, sera, thyroid therapeutics, psychopharmaceuticals, Parkinson’s drugs and other antihyperkinetics, ophthalmics, neuropathy preparations, calcium metabolism regulators, muscle relaxants, narcotics, antilipemics, liver therapeutics, coronary drugs, cardiac drugs, immunotherapeutics, regulatory peptides and their inhibitors, hypnotics, sedatives, gynecological drugs, gout remedies, fibrinolytics, enzyme preparations and transport proteins, enzyme inhibitors, emetics, weight-loss drugs, perfusion promoters, diuretics, diagnostics, corticoids, cholinergics, biliary therapeutics, antiasthmatics, broncholytics, beta-receptor blockers, calcium antagonists, ACE inhibitors, arteriosclerosis remedies, antiphlogistics, anticoagulants, antihypotensives, antihypoglycemics, antihypertensives, antifibrinolytics, antiepileptics, antiemetics, antidotes, antidiabetics, antiarrhythmics, antianemics, antiallergics, anthelmintics, analgesics, analeptics, aldosterone antagonists or antiviral active ingredients or active ingredients for the treatment of HIV infections and AIDS syndrome.

Preference is given to using the inventive copolymers for preparing formulations with active ingredients wherein the active ingredient has a solubility in water under standard conditions (temperature of 23° C. and a pressure of 0.101325 MPa) of less than 0.1 % by weight. Preferably, the solubility of the active ingredient in water under standard conditions is less than 0.05 % by weight.

The formulations can be either real solutions in which both the active ingredient and the inventive copolymer are dissolved in a suitable solvent or mixture of solvents, or solid dispersions in which the active ingredient is embedded in the solid polymer matrix in amorphous form. Solid dispersions are dispersions of one or more active ingredients in a solid polymer matrix [W.L. Chiou, S. Riegelman, Pharmaceutical applications of solid dispersion systems, Journal of Pharmaceutical Sciences, 60 (1971) 1281-1302]. Solid dispersions can be prepared by heating a physical mixture of the active ingredient and the polymer until it melts, followed by cooling and solidification (melting method). Alternatively, solid dispersions can be prepared by dissolving a physical mixture of the active ingredient and the polymer in a common solvent, followed by evaporation of the solvent (solvent method). Solid dispersions may contain the active ingredient molecularly dispersed in a crystalline matrix. Alternatively, solid dispersions may consist of an amorphous carrier; the active ingredient can be either molecularly dispersed in the carrier or form an amorphous precipitate. In any case, the active ingredient needs to be in an amorphous form. “Amorphous” means that less than 5 % by weight of the active ingredient are crystalline.

According to one embodiment of the invention, the solid dispersions according to the invention can be prepared by means of the solvent method. The active ingredient and the polymer are dissolved in organic solvents or solvent mixtures and the solution is then dried. The dissolution can also take place at elevated temperatures (30 - 150° C.) and under pressure. Suitable organic solvents are dimethylformamide, tetrahydrofuran, methanol, ethanol, isopropanol, dimethylacetamide, acetone and/or dioxane or mixtures thereof. These solvents or solvent mixtures may additionally contain up to 20 % by weight of water.

In principle, all types of drying are possible, such as, spray-drying, fluidized-bed drying, drum drying, freeze-drying, vacuum drying, belt drying, roller drying, carrier-gas drying, evaporation etc.

According to another embodiment of the invention, the solid dispersions are prepared by melt processes. The active ingredient is mixed with the polymer. By heating to temperatures of 50 -180° C., the production of the solid dispersion takes place. Here, temperatures above the glass transition temperature of the polymer or the melting point of the active ingredient are advantageous. By adding a softening auxiliary, such as, for example, water, organic solvent, customary organic softeners, it is possible to correspondingly reduce the processing temperature. Of particular advantage are auxiliaries which can afterwards be very easily evaporated off again, i.e., having a boiling point below 180° C., preferably below 150° C.

According to a preferred embodiment, this type of preparation is carried out in a screw extruder. Which process parameters must be individually adjusted here can be determined by those skilled in the art by simple experiments in the scope of his or her conventional specialist knowledge.

According to a preferred embodiment, softeners are added during the melting. Preferred softeners are citric esters such as triethyl citrate or acetyl tributyl citrate, glycol derivatives such as polyethylene glycol, propylene glycol or poloxamers; castor oil and mineral oil derivatives; sebacate esters such as dibutyl sebacate), triacetin, fatty esters such as glycerol monostearate, fatty alcohols such as stearyl alcohol, fatty acids such as stearic acid, ethoxylated oils, ethoxylated fatty acids, ethoxylated fatty alcohols or vitamin E TPGS (tocopherol polyethylene glycol succinate). The softeners may be used in amounts of 0.1 to 40 % by weight, preferably 1 to 20 % by weight, based on the polymer.

The poorly soluble active ingredient is in the amorphous state in the solid dispersion. The absence of crystalline active ingredient can be determined by X-ray diffraction. The so-called “X-ray amorphous” state of the solid dispersions signifies that the crystalline proportion of the active ingredient is less than 5 % by weight.

The amorphous state of the active ingredient can also by investigated with the aid of a DSC thermogram (Differential Scanning Calorimetry). The solid dispersions according to the invention show no active ingredient melting peaks but only a glass transition temperature, which depends also on the type of active ingredient used in the solid dispersions according to the invention. The glass transition temperatures are measured at a heating rate of 20 K/min.

In the course of preparation of the dosage forms according to the invention, customary pharmaceutical auxiliaries may optionally be processed at the same time. These are selected from the class of adsorbents, binders, disintegrants, dyes, fillers, flavorings or sweeteners, glidants, lubricants, preservatives, softeners, solubilizers, solvents or co-solvents, stabilizers (e.g., antioxidants), surfactants, or wetting agents.

The novel polyurethanes inhibit the recrystallization of active pharmaceutical ingredients in the aqueous media of the gastrointestinal tract after release of the active ingredient from the dosage form in which the active ingredient was present in the form of an amorphous solid dispersion of the active ingredient in the polymer matrix of the novel polymer or in the form of a liquid solution of the active ingredient and the inventive polymer in a suitable solvent vehicle system.

### EXAMPLES

### Synthesis Procedure for Inventive Polyurethane IP1

A two-liter glass reactor, equipped with a mechanical stirrer, a condenser, a nitrogen sweep, a thermometer and inlets for the addition of starting materials, was charged with 400 grams of butanone (MEK), 61.7 grams of 2,2-bis(hydroxymethyl)butanoic acid (417 mmol) as component B and 30.0 grams of 1,4-cyclohexane dimethanol (208 mmol) as component C. The resulting mixture was stirred at 100 rpm and heated to 80° C. under a nitrogen atmosphere. Isophorone diisocyanate (138.9 grams, 625 mmol), used as component A, was added within 20 minutes through a dropping funnel. The dropping funnel was rinsed with 50 grams of MEK which was then also added to the reaction mixture. The reaction mixture was stirred for 24 hours at 80° C. under a nitrogen atmosphere. After this, 100 g of water was added, and the resulting mixture was stirred at 70° C. for one hour to hydrolyze residual isocyanate groups. After cooling to ambient temperature, the carboxylic acid groups in the product mixture were neutralized by the addition of 60 grams of a 25% aqueous sodium hydroxide solution (380 mmol). Volatiles were removed and the polymer product was subsequently dried overnight in a vacuum oven at 75° C. at 0.02 MPa.

TABLE 1

Monomers used for polyurethane synthesis

| Monomer | CAS | Abbreviation | Component | MW (g/mol) |
|---|---|---|---|---|
| Isophorone diisocyanate | 4098-71-9 | IPDI | A | 222.3 |
| Tetramethylxylene diisocyanate | 2778-42-9 | TMXDI | A | 244.3 |
| Hexamethylene diisocyanate (not according to the invention) | 822-06-0 | HMDI | Non-cyclic diisocyanate | 168.2 |
| L-Lysine ethyl ester, diisocya-nate (not according to the invention) | 45172-15-4 | LEED | Non-cyclic diisocyanate | 226.2 |
| 2,2-Bis(hydroxymethyl)butyric acid | 10097-02-6 | DMBA | B | 148.2 |
| 2,2-Bis(hydroxymethyl)propionic acid | 4767-03-7 | DMPA | B | 134.1 |
| 1,4-Cyclohexane dimethanol | 105-05-8 | CHDM | C | 144.2 |
| 2,2-Diethyl-1,3-propanediol | 115-76-4 | DEPD | C | 132.2 |
| 2,5-Dimethyl-2,5-hexanediol | 110-03-2 | DMHD | C | 146.2 |
| Isosorbide | 652-67-5 | IS | C | 146.1 |
| Fructose | 57-48-7 | FT | C | 180.2 |
| Xylitol | 87-99-0 | XL | C | 152.1 |
| Neopentyl glycol | 126-30-7 | NPG | C | 104.2 |
| Polyethylene glycol | 25322-68-3 | PEG 400 | Polymericdiol | 400 |

MW = molecular weight.

The other polymers were synthesized by using variations of this procedure. Table 2 describes the differences between the polymerization recipes. Numbers in brackets refer to the used amounts in gram. For the synthesis of IP3, only half the amounts of MEK and water were used. CP2 was prepared using, 140 and 20 grams MEK in the pre-feeding charge and to rinse the dropping funnel respectively, and 35 grams of water to hydrolyze residual isocyanates. In the other cases, the used amounts of solvent and water were identical to the amounts given for the synthesis of IP1. Also reaction times, the temperature during the residual isocyanate hydrolysis and the drying procedure were as described above. In all cases, 90% of the carboxylic acid groups of B were neutralized with a base, after the polymerization and the hydrolysis of residual isocyanate groups. In case of IP6, the polymer was neutralized with triethanolamine instead of an aqueous sodium hydroxide solution.

TABLE 2

Polymer synthesis

| Polymer | Diisocyanate (Component A) | Carboxylic acid diol (Component B) | Component C | Poly. Temp. (°C) | Solvent | Base used for neutralization |
|---|---|---|---|---|---|---|
| IP1 | IPDI (138.9) | DMBA (61.7) | CHDM (30.0) | 80 | MEK | 25% aq. NaOH (60) |
| IP2 | IPDI (138.9) | DMPA (55.9) | CHDM (30.0) | 80 | MEK | 25% aq. NaOH (60) |
| IP3 | IPDI (69.5) | DMBA (30.9) | IS (15.3) | 80 | MEK | 25% aq. NaOH (30) |
| IP4 | IPDI (138.9) | DMBA (61.7) | CHDM (30.0) | 100 | Dioxane | 25% aq. NaOH (60) |
| IP5 | IPDI (138.9) | DMBA (61.7) | DMHD (31.4) | 80 | MEK | 25% aq. NaOH (60) |
| IP6 | IPDI (138.9) | DMBA (61.7) | CHDM (30.0) | 80 | MEK | Triethanolamine (28) |
| IP7 | IPDI (138.9) | DMBA (46.3) | XL (47.6) | 80 | MEK | 25% aq. NaOH (45) |
| IP8 | IPDI (138.9) | DMBA (46.3) | FT (56.3) | 65 | THF | 25% aq. NaOH (45) |
| IP9 | IPDI (138.9) | DMBA (61.7) | DEPD (27.8) | 80 | MEK | 25% aq. NaOH (60) |
| IP10 | TMXDI (140.2) | DMBA (61.7) | CHDM (30.0) | 80 | Dioxane | 25% aq. NaOH (60) |
| IP11 | IPDI (138.9) | DMBA (68.0) | CHDM (23.9) | 80 | Dioxane | 25% aq. NaOH (60) |
| IP12 | IPDI (138.9) | DMBA (61.7) | NPG (21.7) | 80 | Dioxane | 25% aq. NaOH (60) |
| IP13 | IPDI (138.9) | DMBA (72.0) | CHDM (20.1) | 80 | Dioxane | 25% aq. NaOH (70) |
| CP1 | HMDI (105.2) | DMBA (61.7) | CHDM (30.0) | 80 | MEK | 25% aq. NaOH (60) |
| CP2 | LEED (49.0) | DMBA (18.7) | CHDM (13.0) | 80 | MEK | 25% aq. NaOH (18) |
| CP3 | IPDI (138.9) | DMPA (41.9) | PEG 400 (125.0) | 80 | MEK | 25% aq. NaOH (45) |
| CP4 | IPDI (138.9) | DMBA (92.6) | - | 80 | MEK | 25% aq. NaOH (60) |
| CP5 | HMDI (105.2) | DMBA (52.6) | CHDM (39.0) | 80 | Dioxane | 25% aq. NaOH (60) |

MEK = Butanone.

### GPC-Method

Polymer molecular weights were determined by size exclusion chromatography (SEC) at 35° C., using: hexafluoro-2-propanol containing 0.05 wt% of the potassium salt of trifluoroacetic as eluent, narrow molecular weight distribution poly(methyl methacrylate) standards (commercially available from PSS Polymer Standard Solutions GmbH with molecular weights in the range from M = 800 to M = 2,200,000) and a differential refractive index (DRI) detector.

### Preparation of Amorphous Solid Dispersions Via Spray Drying

Danazol-polymer formulations (10 wt% drug loading): Solid dispersions were composed of polymer and danazol. To prepare the formulations, 1.5 g of danazol and 13.5 g of polymer were dissolved in 285 g of methanol (5 wt% solids content). Spray drying was performed on a Büchi Mini Spray Dryer B-290 equipped with a 0.7 mm two-fluid nozzle under the following conditions:

| | |
|---|---|
| Nitrogen flow rate | 35 m³/h |
| Inlet temperature | 85 - 105° C. |
| Outlet temperature | 50 - 70° C. |
| Atomizing pressure | 0.7 MPa |
| Liquid flow rate | 300 g/h |

The product was collected using a cyclone. The drug content of the spray-dried formulations was determined by measuring the UV absorbance at 286 nm; the solid-state properties were analyzed using powder X-ray diffraction (PXRD):

| | |
|---|---|
| Drug content (UV spectroscopy) | 9.4 - 10.8 wt% |
| Solid-state properties (PXRD) | X-ray amorphous |

Amorphous solid dispersions of other drugs were prepared under the same conditions, but in these cases, a 25 wt% drug loading was employed. The drug content in the amorphous solid dispersion was determined by UV spectroscopy by measuring the absorbance at the wave lengths listed in Table 3, and was found to lay between 24.6 and 27.5 wt% in all cases.

TABLE 3

Wavelenghts at which drug absorbance was measured

| Drug | Wavelength (nm) | Drug | Wavelength (nm) |
|---|---|---|---|
| Felodipine | 360 | Celecoxib | 252 |
| Gefitinib | 250 | Telmisartan | 296 |
| Itraconazole | 262 | Fenofibrate | 287 |
| Probucol | 242 | Nifedipine | 360 |

### Preparation of Fasted State Simulated Intestinal Fluid (FaSSIF)

To prepare 1 L of FaSSIF solution, 0.42 g of sodium hydroxide was placed in a volumetric flask and dissolved in approximately 900 mL of water. Then, 3.95 g of sodium dihydrogen phosphate, 6.19 g of sodium chloride, and 2.24 g of FaSSIF/FeSSIF/FaSSGF powder (Biorelevant.com Ltd., London, United Kingdom) were added. The solution was diluted with water to 1 L, the pH was adjusted to 6.8 using 1 molar aqueous sodium hydroxide solution and allowed to stand for 2 h.

### Dissolution Testing

In vitro dissolution tests were done to quantify the drug release and measure the maintenance of supersaturation. To this end, 300 ml FaSSIF were filled into the dissolution vessels of an ERWEKA dissolution tester with mini glass vessels (stirring speed approximately 75 rpm). After a temperature of 37° C. had been reached, a defined amount of the spray-dried formulation (equivalent to a drug concentration of 0.14 mg/ml) was added. Samples of 3 mL were withdrawn after 5 min, 15 min, 30 min, 60 min, 90 min, 120 min, 180 min, 240 min, 300 min and 360 min. All samples were filtered through 0.45 µm PVDF syringe filters and diluted with methanol or methanol/water (1:4 or 1:10, depending on the drug concentration). The concentration of the drug in solution was determined by UV spectroscopy using a calibration curve of the pure drug in methanol. To evaluate the performance of the polymer, the area under the concentration-time curve (AUC) was calculated as follows:

$$A U {C}_{0 \to {t}_{n}} = \frac{1}{2} {\sum }_{i = 1}^{n - 1} \left({C}_{{t}_{i}} + {C}_{{t}_{i + 1}}\right) ⋅ \left({t}_{i + 1} - {t}_{i}\right)$$

$${\text{C}}_{\text{t}} = \text{Concentration at time t in} \text{mg} / \text{mL} \text{, t=} \text{Time in min}$$

The AUC value was used to calculate active pharmaceutical ingredient (API) release as a percentage value of maximum possible API release. Maximum release meaning complete dissolution of the used amount of API for the entire duration of the 6 hours (360 minutes) dissolution experiment.

$$\text{API release =} \frac{A U C \left(m g / m l ⋅ m i n\right)}{360 \left(m i n\right) ⋅ C m a x \left(m a / m l\right)} ⋅ 100 \%$$

The results summarized in Table 4 and 5 show that the inventive polymers IP are effective crystallization inhibitors.

TABLE 4

Crystallization inhibition performance of synthesized polymers

| Polymer | Mₙ (g/mol)* | M_{w} (g/mol)* | Danazol release (%) |
|---|---|---|---|
| IP1 | 4 060 | 12 600 | 94 |
| IP2 | 4 310 | 12 300 | 99 |
| IP3 | 3 140 | 8 490 | 76 |
| IP4 | 5 700 | 22 700 | 84 |
| IP5 | 4 030 | 10 500 | 96 |
| IP6 | 3 570 | 8 410 | 96 |
| IP7 | 4 010 | 32 200 | 76 |
| IP8 | 4 080 | 19 200 | 84 |
| IP9 | 3 650 | 9 770 | 85 |
| IP10 | 4 110 | 10 900 | 68 |
| IP11 | 5 700 | 22 700 | 93 |
| IP12 | 5 630 | 16 000 | 82 |
| IP13 | 6 970 | 20 300 | 82 |
| CP1 | 4 420 | 16 500 | 24 |
| CP2 | 3 990 | 15 200 | 20 |
| CP3 | 25 500 | 65 700 | 14 |
| CP4 | 4 860 | 13 900 | 20 |
| CP5 | 5 380 | 22 300 | 33 |

*Determined by GPC.

TABLE 5

Release of other drugs in combination with inventive polyurethanes

| Drug | Polyurethane | Drug release (%) |
|---|---|---|
| Felodipine | IP1 | 94 |
| Gefitinib | IP13 | 100 |
| Itraconazole | IP1 | 51 |
| Probucol | IP13 | 100 |
| Celecoxib | IP1 | 95 |
| Telmisartan | IP1 | 98 |
| Fenofibrate | IP1 | 54 |
| Nifedipine | IP1 | 96 |

1. An isocyanate free polyurethane comprising components A to D with 45-70 wt% of at least one diisocyanate A with at least one ring in the molecular structure between the two isocyanate groups; with 15-40 wt% of at least one component B with i) two primary hydroxy groups, ii) one secondary or tertiary carboxylic acid group iii) a molecular weight between 100 and 250 g/mol and iv) no additional group reactive towards an isocyanate; with 5-30 wt% of at least one component C with i) at least two hydroxy groups, ii) a molecular weight between 60 and 250 g/mol, iii) no acid group and iv) no primary or secondary amine or thiol group and optionally with 0-5 wt% of one or more components D which i) contain one or two groups reactive toward isocyanate groups and ii) maximum one of these reactive groups is a hydroxy group, whereby a total amount of incorporated components A to D adds up to 100% by weight.

2. The polyurethane according to claim 1, wherein the diisocyanate A is isophorone diisocyanate.

3. The polyurethane according to claim 1, wherein the component B is 2,2-bis(hydroxymethyl)butyric acid.

4. The polyurethane according to claim 1, wherein the component B is 2,2-bis(hydroxymethyl)propionic acid.

5. The polyurethane according to claim 1 wherein the component C is 1,4-cyclohexane dimethanol.

6. The polyurethane according to claim 1, wherein the component C is neopentyl glycol.

7. The polyurethane according to claim 1, wherein the component C is isosorbide.

8. The polyurethane according to claim 1, wherein the component C is 2,5-dimethyl-2,5-hexanediol.

9. The polyurethane according to claim 1, wherein the component C is 2,2-diethyl-1,3-propanediol.

10. (canceled)

11. The polyurethane according to claim 1, wherein a weight average molecular weight of the polyurethane is in the range of 2,000 to 100,000 g/mol.

12. The polyurethane according to claim 1, wherein at least 25% of the carboxylic acid groups of component B are neutralized.

13. An isocyanate free polyurethane comprising components A to C with 50-65 wt% of isophorone diisocyanate as A; with 20-35 wt% of at least one component B selected from the group consisting of 2,2-bis(hydroxymethyl)butyric acid and 2,2-bis(hydroxymethyl)propionic acid, and with 5-30 wt% of at least one component C selected from the group consisting of 1,4-cyclohexane dimethanol, 1,3-cyclohexane dimethanol, 2,2-diethyl-1,3-propanediol, 2,5-dimethyl-2,5-hexanediol, isosorbide, and neopentyl glycol, whereby the total amount of incorporated components A to C adds up to 100% by weight.

14. The polyurethane according to claim 13, wherein at least 25% of the carboxylic acid groups of component B) are neutralized.

15. The polyurethane according to claim 13, wherein a ratio between isocyanate groups of diisocyanate A and sum of primary and secondary alcohol groups of B and C is 1.1:1 to 0.9:1.

16. The polyurethane according to claim 13, wherein a weight average molecular weight of the polyurethane is in the range of 4,000 to 60,000 g/mol.

17. A pharmaceutical dosage form, comprising a polyurethane according to claim 1 and an active pharmaceutical ingredient with a solubility in water at standard conditions of less than 0.1 % by weight, wherein the active ingredient is present in an amorphous form.

18. A method for inhibiting recrystallization of an active ingredient in a pharmaceutical dosage form in an aqueous environment of a human or animal body, wherein the active ingredienthas a solubility in water at standard conditions of less than 0.1 % by weight, and wherein the active ingredient is present in the pharmaceutical dosage form in the amorphous form comprising adding a polyurethane according to claim 1 to the pharmaceutical dosage form.

19. A method for inhibiting recrystallization of an agricultural active ingredient in an agricultural dosage form in soil, whereby the active ingredient has a solubility in water at standard conditions of less than 0.1 % by weight, and wherein the active ingredient is present in the agricultural dosage forms in the amorphous form comprising adding a polyurethane according to claim 1 to the agricultural dosage form.
